(19) United States
(12) Patent Application Publication
(10) Pub. No.: US 2023/0293905 A1
(43) Pub. Date: Sep. 21, 2023

(54) LIGHT IRRADIATION SYSTEM, WEARABLE DEVICE, INSTALLATION-TYPE LIGHT IRRADIATION DEVICE, AND LIGHT IRRADIATION METHOD

(71) Applicant: KYOCERA Corporation, Kyoto-shi, Kyoto (JP)

(72) Inventor: Nobuko KUSHIDA, Higashiyodogawa-ku, Osaka (JP)

(21) Appl. No.: 18/023,131

(22) PCT Filed: Aug. 26, 2021
(86) PCT No.: PCT/JP2021/031307

§ 371 Date: Feb. 24, 2023

(30) Foreign Application Priority Data
Aug. 27, 2020 (JP) 2020-143867

(51) Int. Cl. A61N 5/06; H05B 47/16; H05B 47/11

(52) U.S. Cl. CPC A61N 5/0622; H05B 47/11; H05B 47/16; A61N 2005/0628; A61N 2005/0648; A61N 2005/0663; A61N 2005/0667

(57) ABSTRACT

Provided is a light irradiation system and a light irradiation device that irradiate an eye of a subject with light having a predetermined wavelength band to produce a non-visual effect in mind and body of the subject. A light irradiation system according to an aspect of the present disclosure includes an irradiator configured to irradiate an eye of a subject with irradiation light having a predetermined wavelength band, and a shielding mechanism configured to decrease illuminance of light different from the irradiation light or decrease illuminance of light included in light different from the irradiation light and having a wavelength band different from the predetermined wavelength band.

### TECHNICAL FIELD

The present disclosure relates to a light irradiation system, a wearable device, an installation-type light irradiation device, and a light irradiation method.

### BACKGROUND OF INVENTION

Ganglion cells, such as intrinsically photosensitive retinal ganglion cells (iPRGC), are present in eyes, which can receive light but are not directly related to visual function.

Light reception by the intrinsically photosensitive retinal ganglion cells is known to produce various non-visual effects in mind and body. For example, Non-Patent Document 1 reports that exposure to light having a wavelength band corresponding to green light alleviates acute and chronic pain.

### CITATION LIST

### Non-patent Literature

Non-Patent Document 1: Mohab M. Ibrahima et, al., “Long-lasting antinociceptive effects of green light in acute and chronic pain in rats”, Pain. Vol. 158, No. 2, p. 347-360, 2017

### SUMMARY

In an aspect of the present disclosure, a light irradiation system includes an irradiator configured to irradiate an eye of a subject with irradiation light having a predetermined wavelength band, and a shielding mechanism configured to decrease illuminance of light included in light different from the irradiation light and having a wavelength band different at least from the predetermined wavelength band.

In an aspect of the present disclosure, a wearable device is wom on the head of a subject, and includes an irradiator configured to irradiate an eye of the subject with irradiation light having a predetermined wavelength band, and a shielding mechanism configured to decrease illuminance of light reaching the eye of the subject, included in light different from the irradiation light and having a wavelength band different at least from the predetermined wavelength band.

In an aspect of the present disclosure, an installation-type light irradiation device includes a support configured to support a user in a sitting or lying position, an irradiator configured to irradiate an eye of a subject with irradiation light having a predetermined wavelength band, and a shielding mechanism configured to decrease illuminance of light reaching the eye of the subject, included in light different from the irradiation light, and having a wavelength band different at least from the predetermined wavelength band, in which the irradiator is installed in the shielding mechanism at a position opposite to the eye of the subject.

According to an aspect of the present disclosure, a light irradiation method includes calibrating by
irradiating an eye of a subject with irradiation light having a predetermined intensity for a certain period of time,
acquiring a first intensity of return light detected when an eyelid of the subject is in an open state and a second intensity of the return light detected when the eyelid of the subject is in a closed state, and
determining in advance a first reference intensity of the irradiation light when the eyelid of the subject is in the open state and a second reference intensity of the irradiation light when the eyelid of the subject is in the closed state based on a comparison result between the first intensity and the second intensity;
emitting light having the first reference intensity;
changing the intensity of the irradiation light from the first reference intensity to the second reference intensity upon determination as to whether the eyelid of the subject is in the closed state and whether predetermined time has passed in the closed state; and changing the intensity of the irradiation light from the second reference intensity to the first reference intensity upon determination as to whether the eyelid of the subject is in the open state.

### BRIEF DESCRIPTION OF THE DRAWINGS

FIG. 1 is a block diagram illustrating an example structure of main components of a light irradiation system according to a first embodiment of the present disclosure.

FIG. 2 is a perspective view illustrating an example structure of a light irradiation system according to an aspect of the present disclosure.

FIG. 3 is a schematic diagram showing an example of temporal changes in the intensity of return light received by a light receiver and the intensity of irradiation light emitted by an irradiator.

FIG. 4 is a flowchart illustrating the process flow of the light irradiation system according to the first embodiment of the present disclosure.

FIG. 5 is a perspective view illustrating an example structure of a light irradiation system according to a second embodiment of the present disclosure.

FIG. 6 is a bottom view of an example structure of the light irradiation system according to the second embodiment of the present disclosure.

### DESCRIPTION OF EMBODIMENTS

### First Embodiment

An embodiment of the present disclosure will be described in detail below. Unless otherwise specified in the present specification, “A to B” representing a numerical value range means “A or more and B or less”.

### Structure of Light Irradiation System 100

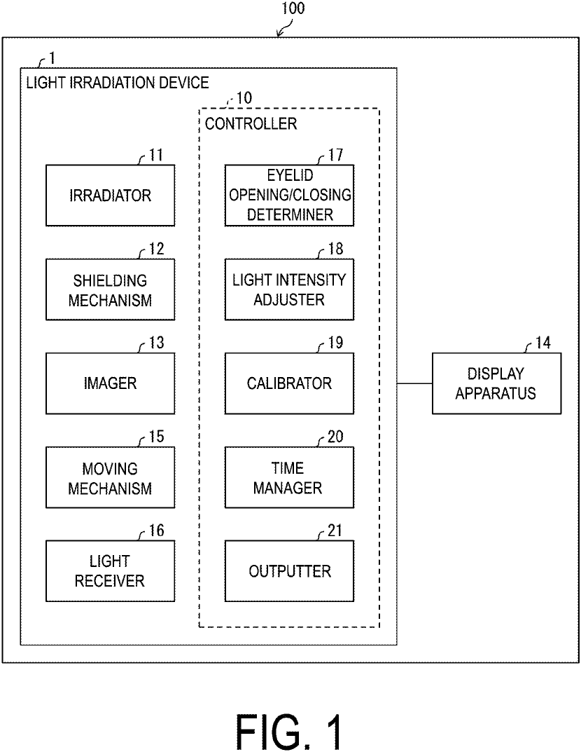

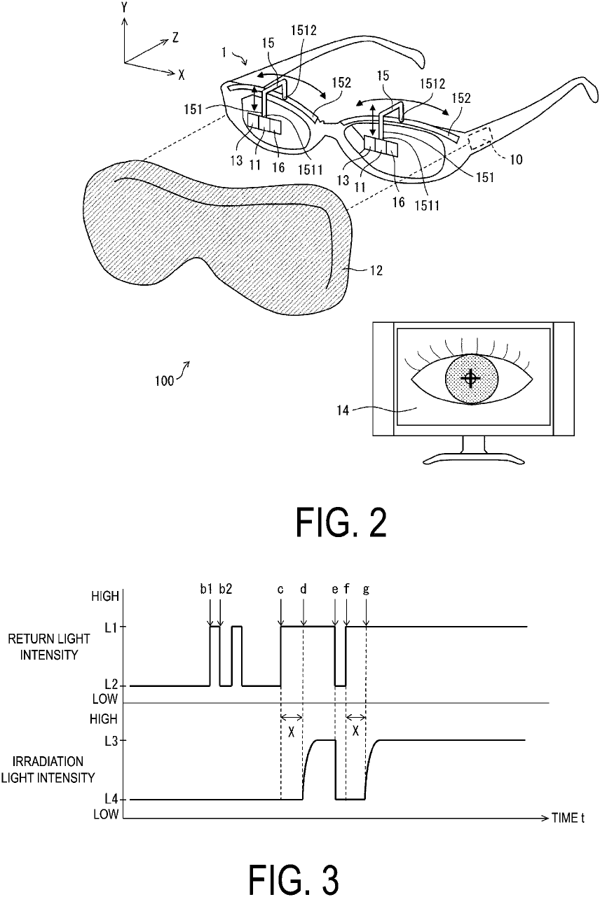

The structure of a light irradiation system 100 is described with reference to FIG. 1 and FIG. 2. FIG. 1 is a block diagram illustrating an example structure of main components of the light irradiation system 100 according to a first embodiment of the present disclosure. FIG. 2 illustrates an example structure of the light irradiation system 100 according to an aspect of the present disclosure.

The light irradiation system 100 includes a light irradiation device 1 that irradiates eyes of a subject with irradiation light having a predetermined wavelength band. The light irradiation device 1 may be an installation-type device which is not easily carried, or may be a wearable device.

In addition to the light irradiation device 1, the light irradiation system 100 may further include a display 14 that displays images and various types of information acquired from the light irradiation device 1. Hereinafter, the light irradiation system 100 with the display 14 is described as an example.

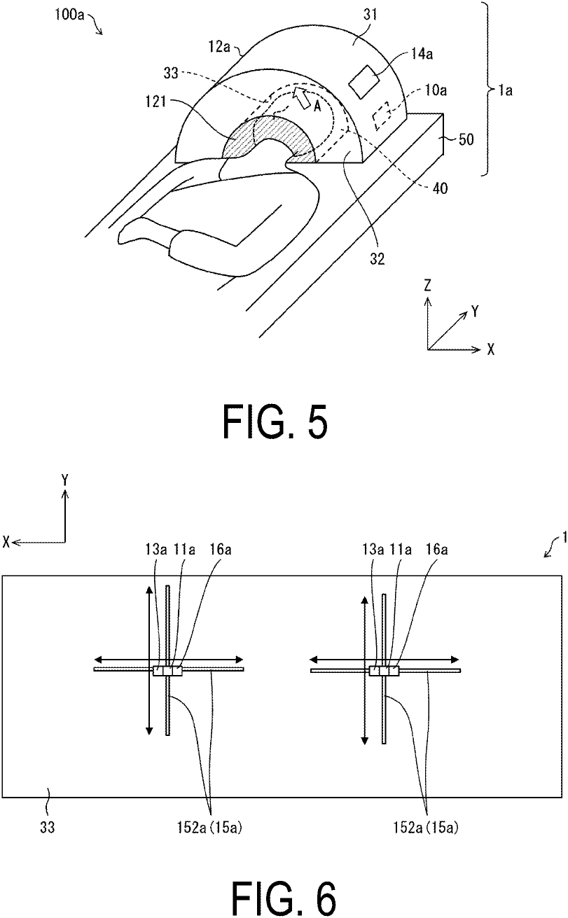

In the case of an installation-type device as the light irradiation device 1 (see FIG. 5), the light irradiation device 1 includes a support 50 that supports the subject in a sitting or lying position. The support 50 may be, for example, a chair or a bed. The installation-type light irradiation device 1 will be described later with a specific example. In that case, the display 14 may be integrated with the light irradiation device 1. For example, the display 14 may be disposed on the light irradiation device 1.

On the other hand, in the case of a wearable device as the light irradiation device 1 (see FIG. 2), the light irradiation device 1 may be in any shape that can be wom on the head of the subject (for example, an eyeglass shape, a goggle shape, or a headgear shape). In this case, the light irradiation device 1 may include a wearing mechanism for attaching the light irradiation device 1 to the body of the subject. For example, in the case of the eyeglass-type light irradiation device 1, the wearing mechanism may be nose pads and temples (so-called “temples” hung on the ears) or the like. The wearing mechanism in the case of the goggle-type light irradiation device 1 may be a head strap (fixing band) or the like. As illustrated in FIG. 2, when the light irradiation device 1 is a wearable device, the display 14 may be provided separately from the light irradiation device 1. For example, the display 14 may be a display unit or a display of a computer (not illustrated) that is communicable with the light irradiation device 1.

As illustrated in FIG. 1, the light irradiation device 1 includes an irradiator 11 and a shielding mechanism 12. The light irradiation device 1 may further include an imager 13, a moving mechanism 15, a light receiver 16, and a controller 10, although not essential. FIG. 1 and FIG. 2 illustrate the light irradiation device 1 including the imager 13, the moving mechanism 15, the light receiver 16, and the controller 10 in addition to the irradiator 11 and the shielding mechanism 12. The light irradiation device 1 may also include a storage (not illustrated) that stores various computer programs read by the controller 10 and data for use in various processes executed by the controller 10.

According to an aspect of the present disclosure, a light irradiation system, a wearable device, and an installation-type light irradiation device can produce a non-visual effect in mind and body of a subject by irradiating the eyes of the subject with light having a predetermined wavelength band.

An example will be given of a case in which the light irradiation device 1 is a wearable eye-glass type.

### Irradiator 11

The irradiator 11 includes one or more light sources for irradiating eyes of a subject with irradiation light having a predetermined wavelength band. The irradiator 11 may include, for example, a light emitting element such as a light emitting diode (LED) or a semiconductor laser (LD) as a light source. By using such a light-emitting element, the irradiator 11 can selectively irradiate monochromatic light having a narrow emission wavelength width. Alternatively, the irradiator 11 may include a light source that emits white light and an optical filter (for example, a band-pass filter) that transmits only the light having a predetermined wavelength band. By selecting and using an appropriate optical filter, the irradiator 11 can selectively emit irradiation light having a desired wavelength band.

The light irradiation device 1 illustrated in FIG. 2 can irradiate both eyes of the subject with light having a predetermined wavelength band, but is not limited thereto. The light irradiation device 1 may irradiate one eye of the subject with light having a predetermined wavelength band. In that case, only one light source of the irradiator 11 may be turned on. Alternatively, the irradiator 11 may be detachably attached to the light irradiation device 1, and the irradiator 11 attached to the unused side can be removed.

As illustrated in FIG. 2, the irradiator 11 may be attached to the moving mechanism 15 which will be described later, and the position of the irradiator 11 with respect to the position of the eye of the subject may be changeable using the moving mechanism 15.

### Wavelength Band of Irradiation Light

The irradiation light may have a wavelength band that is expected to improve mental and physical conditions of the subject when the irradiation light is emitted to the eyes of the subject.

Irradiating the eyes of the subject with green light can continuously alleviate body pain, such as an aching pain felt by the subject, after the irradiation. Therefore, the wavelength band of the irradiation light may be the wavelength band of green light. In this case, the wavelength band of the irradiation light is, for example, from 450 nm to 600 nm, and may be from 500 nm to 550 nm. The irradiation light may be green light with its peak top at 525 nm.

Irradiating the eyes of the subject with violet light can reduce the risk of loss of acuity of vision or decrease the rate of progression of myopia of the eyes of the subject. Therefore, the wavelength band of the irradiation light may be the wavelength band of violet light. In this case, the wavelength band of the irradiation light may be from 350 nm to 410 nm.

The symptoms of jetlag of the subject can be alleviated by irradiating the eyes of the subject with blue light. Therefore, the wavelength band of the irradiation light may be the wavelength band of blue light. However, when the eyes of the subject are irradiated with blue light, an optical filter that blocks ultraviolet rays and near-ultraviolet rays may also be provided reducing the damage to the eyes of the subject as much as possible.

When the effect of improving the mental and physical conditions of the subject is expected, the irradiation light may be continuous light that is continuously emitted to the eyes of the subject, or pulsed light that is emitted intermittently.

### Shielding Mechanism 12

The light irradiation device 1 according to the present disclosure includes the shielding mechanism 12. This decreases the illuminance of light reaching the eyes of the subject and having a wavelength band at least different from the wavelength band of the irradiation light, when the eyes of the subject are irradiated with the irradiation light having a predetermined wavelength band. For example, irradiating the eyes of the subject with green light can sustain the pain relieving effect after irradiation. The light irradiation device 1 including the shielding mechanism 12 can improve the pain relieving effect obtained by irradiating the eyes of the subject with green light.

The shielding mechanism 12 may decrease the illuminance of light different from the irradiation light, or the illuminance of light included in light different from the irradiation light and having a wavelength band different from the predetermined wavelength band. As used herein, the light different from the irradiation light may be, for example, sunlight or light from indoor lighting.

The shielding mechanism 12 may be made of a light shielding fabric or resin, or a metal having a light shielding or light reflecting characteristic. Alternatively, the shielding mechanism 12 may include an optical filter that selectively transmits light having a predetermined wavelength band and blocks light having a wavelength band different from the predetermined wavelength band. In the former case, the shielding mechanism 12 can decrease the illuminance of light from the outside of the light irradiation device 1 over the entire wavelength bands. In the latter case, the shielding mechanism 12 can decrease the illuminance of light included in the light from the outside of the light irradiation device 1 and having a wavelength band different from the predetermined wavelength band. This decreases the illuminance of light reaching the eyes of the subject and having the wavelength band different from the wavelength band of the irradiation light when the eyes of the subject are irradiated with the irradiation light having the predetermined wavelength band. When the shielding mechanism 12 is made of a light reflecting material, the light from the outside of the light irradiation device 1 can be reduced, while the light inside the light irradiation device 1 can be reflected to an irradiation target.

The shielding mechanism 12 may be disposed in a manner that no gap exists between the subject wearing the light irradiation device 1 and the light irradiation device 1. The shielding mechanism 12 may be integrated with or may be separated from the light irradiation device 1.

As illustrated in FIG. 2, the shielding mechanism 12 may have a goggle-like shape covering the entire frame portion of the light irradiation device 1. In this case, the shielding mechanism 12 can cover the frame portion of the light irradiation device 1. This avoids light from the outside of the light irradiation device 1 from entering through the gap between the forehead, temples, cheeks, and nose of the subject and the light irradiation device 1, and reaching the eyes of the subject. Alternatively, the shielding mechanism 12 may cover the entire head of the subject. Alternatively, the subject wearing the light irradiation device 1 illustrated in FIG. 2 or the head portion of the subject may be covered with another shielding mechanism 12.

The shielding mechanism 12 illustrated in FIG. 2 shields light reaching both eyes of the subject from the outside of the light irradiation device 1, but is not limited thereto. For example, the light irradiation device 1 may include the shielding mechanism 12 for the right eye and the shielding mechanism 12 for the left eye. In that case, when one eye of the subject is irradiated with the irradiation light, only the shielding mechanism 12 on this side may be used. With this structure, the field of view of the eye on the side not irradiated with the irradiation light is not blocked by the shielding mechanism 12. This allows the subject to visually recognize the surrounding situation with the eye on the side not irradiated with the irradiation light.

### Imager 13

The imager 13 is a camera for capturing an image of the subject, and may be a digital camera or a digital video camera. The imager 13 may capture an image of the face of the subject irradiated with the irradiation light, or may capture an image of the eyes and surrounding areas of the subject irradiated with the irradiation light.

Image data (still image data or moving image data) captured by the imager 13 may be transmitted from the light irradiation device 1 to the display 14.

The imager 13 may be disposed at any position where the imager 13 can capture the image of the eyes and surrounding areas of the subject irradiated with the irradiation light. In an example, as illustrated in FIG. 2, the imager 13 may be disposed near the irradiator 11. In this case, the position of the imager 13 may be changed together with the irradiator 11 using the moving mechanism 15.

### Moving Mechanism 15

The moving mechanism 15 receives the operation for changing the position of the irradiator 11 with respect to the position of the eye (in particular, the pupil) of the subject. The moving mechanism 15 is provided to move the position of the irradiator 11 from which light is emitted toward the eye, or in particular the pupil, of the subject.

The moving mechanism 15 may have any structure with which the position of the irradiator 11 can be changed with respect to the position of the pupil of the eye of the subject. As an example, as illustrated in FIG. 2, the moving mechanism 15 may include a holding portion 151 that holds the irradiator 11 at a first end 1511, and a guide rail 152 for moving the holding portion 151 in the X-axis direction (left-right direction). Hereinafter, a direction parallel to the front-rear axis of the eyeball (that is, the axis connecting the cornea and retina) is referred to as the Z-axis direction, a direction parallel to the left-right axis (that is, the axis connecting the left ear and the right ear) is referred to as the X-axis direction, and a direction parallel to the vertical axis (that is, the axis connecting the head and feet) is referred to as the Y-axis direction.

The guide rail 152 may be, for example, a groove formed in an upper portion of a frame of the light irradiation device 1. A second end 1512 of the holding portion 151 opposite to the first end 1511 may slidably abut an inner surface of the groove. Thus, the holding portion 151 can move in the X-axis direction along the guide rail 152.

The moving mechanism 15 may move the position of the irradiator 11 in the Y-axis direction (vertical direction). For example, the holding portion 151 may have a structure expandable and contractable in the Y-axis direction. Alternatively, a fixing member (not illustrated) that fixes the irradiator 11 to the holding portion 151 may be provided slidably along the holding portion 151 along which the irradiator 11 can move.

For example, a medical worker such as a doctor, a nurse, and the like who is in charge of the subject (or the subject himself/herself) can manually change the position of the irradiator 11 along the guide rail 152. The position of the irradiator 11 may be automatically changed along the guide rail 152 based on the image data of the imager 13.

With the moving mechanism 15, the light irradiation device 1 can accurately irradiate the eye of the subject, particularly the pupil, with the irradiation light and cause the irradiation light to reach the central fovea of the retina.

### Light Receiver 16

The light receiver 16 detects intensity of return light of the irradiation light. The return light means the light reflected on the surface of the eyelid or the like and returns to the irradiator 11 when the irradiation light is emitted by the irradiator 11 to the eye or eyelid of the subject. The light receiver 16 may be disposed at any position where the return light can be received. The light receiver 16 may include one or more light receiving elements. The light receiver 16 may include a plurality of light receiving elements with which the intensity of the return light can be detected with higher accuracy. As an example, the light receiving element is an avalanche photodiode. For example, an intensity signal outputter (not illustrated) may be connected to the light receiver 16, and the intensity signal outputter may output an intensity signal representing the intensity of the reflected light detected by the light receiver 16.

The light receiver 16 may be disposed at any position where the return light can be received. In an example, the light receiver 16 may be disposed in the vicinity of the irradiator 11 as illustrated in FIG. 2. In this case, the position of the light receiver 16 may be changed together with the irradiator 11 using the moving mechanism 15.

### Display 14

The display 14 displays images captured by the imager 13. The display 14 may be, for example, a liquid crystal display or an organic EL display attached to a terminal operated by the medical worker. The display 14 and the imager 13 may be connected to each other by wire or wirelessly, and the image captured by the imager 13 may be displayed on the display 14 in real time.

The display 14 may display the image of the subject captured by the imager 13. Based on the image displayed on the display 14, the medical worker can know the position irradiated with the irradiation light and an open/closed state of the eyelid of the subject.

### Controller 10

The controller 10 controls the light irradiation device 1 to perform the processing of each function. In an example, the controller 10 may be a CPU included in the light irradiation device 1. The controller 10 includes an eyelid opening/closing determiner 17, a light intensity adjuster 18, a calibrator 19, a time manager 20, and fan outputter 21.

The light irradiation system 100 can irradiate the eyes of the subject with the irradiation light having the predetermined wavelength when the eyes are closed. However, the illuminance of the irradiation light when the light passes through an eyelid and reaches the retina of the subject differs depending on whether the eyelid is in an open state or a closed state. The light irradiation system 100 may include the controller 10 to allow adjustment of the illuminance of the irradiation light in accordance with the open/closed state of the eyelid of the subject.

### Eyelid Opening/Closing Determiner 17

The intensity of the return light detected when the eyelid of the subject is in the closed state is higher than the intensity of the return light detected when the eyelid is in the open state. This is because the irradiation light is reflected on the surface of the eyelid of the subject.

The eyelid opening/closing determiner 17 determines the open/closed state of the eyelid of the subject based on the intensity of the return light received by the light receiver 16. The eyelid opening/closing determiner 17 may determine the open/closed state of the eyelid of the subject, and may also measure time during which the open state and the closed state of the eyelid of the subject continues.

### Light Intensity Adjuster 18

When the eyelid of the subject is in the closed state, the light intensity adjuster 18 may increase the intensity of the irradiation light emitted by the irradiator 11 to be greater than the intensity of the irradiation right when the eyelid of the subject is in the open state, based on the determination result by the eyelid opening/closing determiner 17.

The light intensity adjuster 18 may adjust the intensity of the irradiation light by applying a first reference intensity (for example, see intensity L4 illustrated in FIG. 3) and a second reference intensity (for example, see intensity L3 in FIG. 3) determined in advance through calibration process performed by the calibrator 19 which will be described later. As used herein, the first reference intensity is the intensity of irradiation light applied when the eyelid of the subject is in the open state, and the second reference intensity is the intensity of irradiation light applied when the eyelid of the subject is in the closed state. The first reference intensity and the second reference intensity may be determined for each subject.

Since the eyelid is closed for a very short time when the subject blinks, it is not necessary to change the intensity of the irradiation light for each blink. The light intensity adjuster 18 may not increase the intensity of the irradiation light when the closed state of the eyelid of the subject continues for less than a predetermined period of time. The predetermined period of time during which the closed state of the eyelid of the subject continues can be preset, for example, to any fixed value in seconds (for example, one second).

The eye of the subject continues to be exposed to the high irradiation light when the eye of the subject is closed for the predetermined period of time or longer. When the subject then opens his/her eyelids, the eye of the subject is irradiated with the irradiation light that remains at the same intensity as when the eyelid is closed. This may cause the subject to feel glaring. The light intensity adjuster 18 may decrease the intensity of the irradiation light when the eyelid of the subject is in the open state following the closed state for the predetermined period of time or longer.

### Processing Example of Eyelid Opening/Closing Determiner 17 and Light Intensity Adjuster 18

Processing performed by the eyelid opening/closing determiner 17 and the light intensity adjuster 18 is described with reference to FIG. 3. FIG. 3 is a schematic diagram showing an example of temporal changes in the intensity of the returned light received by the light receiver 16 and the intensity of the irradiation light emitted by the irradiator 11.

The intensity of the irradiation light and the intensity of the return light are constant until time point b1 in FIG. 3. During this time, the eyelid opening/closing determiner 17 determines that the eyelid of the subject is in the open state, and the irradiator 11 emits irradiation light having a predetermined intensity (for example, 4 Lux to 100 Lux). With the intensity of the irradiation light of 4 Lux to 100 Lux, body pains such as aching pains felt by the subject can continuously be alleviated without causing the subject to feel glaring. It is assumed that predetermined time X during which the closed state of the eyelid of the subject continues is set in advance to five seconds as an example.

At time point b1, the intensity of the return light increases from intensity L2 to intensity L1, and returns to the intensity L2 at time point b2. The eyelid opening/closing determiner 17 determines that the eyelid of the subject is in the closed state at the time point b1, and starts measuring the time during which the closed state of the eyelid continues. The eyelid opening/closing determiner 17 determines that the eyelid of the subject is in the open state at the time point b2. The eyelid opening/closing determiner 17 also measures the time from the time point b1 to the time point b2 as the time during which the closed state of the eyelid of the subject continues.

The light intensity adjuster 18 compares the time measured by the eyelid opening/closing determiner 17 (the time during which the closed state of the eyelid of the subject continues) with the predetermined time X (five seconds) set in advance. When the time during which the closed state of the eyelid of the subject continues is shorter than five seconds set as the predetermined time X, the light intensity adjuster 18 does not change the intensity of the irradiation light and maintains the intensity at the intensity L4 (first reference intensity). Thus, the light intensity adjuster 18 does not change the intensity of the irradiation light when the subject blinks and the eyelid is closed for a very short time.

At time point c, the intensity of the return light increases again from the intensity L2 to the intensity L1. The eyelid opening/closing determiner 17 determines that the eyelid of the subject is in the closed state at the time point c, and starts measuring the time during which the closed state of the eyelid continues. The eyelid opening/closing determiner 17 determines that the eyelid of the subject is in the closed state at time point d. The eyelid opening/closing determiner 17 also measures the time from the time point c to the time point d as the time during which the closed state of the eyelid of the subject continues.

The light intensity adjuster 18 compares the time measured by the eyelid opening/closing determiner 17 (the time during which the closed state of the eyelid of the subject continues) with the predetermined time X. The light intensity adjuster 18 changes the intensity of the irradiation light from the intensity L4 to the intensity L3 (second reference intensity) when the time during which the closed state of the eyelid of the subject continues is longer than the predetermined time X. Accordingly, when the closed state of the eyelid of the subject continues longer than the predetermined time X (for example, when the subject falls asleep), the light intensity adjuster 18 increases the intensity of light emitted by the irradiator 11. This allows the light intensity adjuster 18 to adjust the intensity of the irradiation light to avoid a significant drop in the illuminance of the irradiation light reaching the retina of the subject, even when the subject being irradiated with the irradiation light falls asleep, for example, during irradiation.

Time point e corresponds to the time when, for example, the subject wakes up and opens his/her eyelid. That is, at the time point e, the intensity of the return light decreases again from the intensity L1 to the intensity L2. At the time point e, the eyelid opening/closing determiner 17 determines that the eyelid of the subject is in the open state, and starts measuring the time during which the open state of the eyelid continues. The eyelid opening/closing determiner 17 immediately decreases the intensity of light emitted by the irradiator 11. As described above, the light intensity adjuster 18 decreases the intensity of the irradiation light from the intensity L3 to the intensity L4 when the eyelid of the subject is in the open state subsequent to the closed state continued for the predetermined time or longer. This avoids the irradiation of the eye of the subject when awakens with the irradiation light having the high intensity.

At time point f, the intensity of the return light increases again from the intensity L2 to the intensity L1. At the time point f, the eyelid opening/closing determiner 17 determines that the eyelid of the subject is in the closed state and starts measuring the time during which the closed state of the eyelid continues. At time point g at which the predetermined time X has elapsed from the time point f, the light intensity adjuster 18 changes the intensity of the light emitted by the irradiator 11 from the intensity L4 to the intensity L3.

### Calibrator 19

Referring back to FIG. 1, the calibrator 19 performs processing for determining the first reference intensity and the second reference intensity described above. Specifically, the calibrator 19 continuously irradiates the eye of the subject with the irradiation light having a predetermined intensity for a certain period of time and acquires a first intensity of the return light detected when the eyelid of the subject is in the open state and a second intensity of the return light detected when the eyelid of the subject is in the closed state. The calibrator 19 may determine in advance the first reference intensity of the irradiation light when the eyelid of the subject is in the open state and the second reference intensity of the irradiation light when the eyelid of the subject is in the closed state, based on the comparison result between the first intensity and the second intensity.

The first intensity is the intensity of the return light detected by the light receiver 16 when the eyelid of the subject is in the open state, and the second intensity is the intensity of the return light detected by the light receiver 16 when the eyelid of the subject is in the closed state. The first reference intensity is the intensity of the irradiation light emitted from the irradiator 11 to the subject when the eyelid of the subject is in the open state, and the second reference intensity is the intensity of the irradiation light emitted by the irradiator 11 to the subject when the eyelid of the subject is in the closed state.

The illuminance of the irradiation light that reaches the retina of the subject when the subject closes the eyelid varies depending on the state of the eyelid of the subject. The state of the eyelid corresponds to, for example, a thickness of the eyelid or the presence or absence of a substance present on the eyelid surface. Since the thickness of the eyelid differs for each subject, the illuminance of the irradiation light reaching the retina of the subject differs for each subject. Therefore, the first reference intensity and the second reference intensity may be determined for each subject.

The calibrator 19 may determine the first reference intensity for each subject. The conditions of the cornea, the crystalline lens, the eyeball, the iris, and the like are known to differ between individuals. For example, a curvature of the cornea, a curvature of the crystalline lens, a length of the ocular axis of the eyeball, a regulatory power of the iris, and a degree of opacity of the crystalline lens are different for each subject. Accordingly, with the irradiation light having the same intensity, the illuminance of the irradiation light reaching the retina may be different for each subject. The calibrator 19 may therefore determine the first reference intensity with which each subject is irradiated based on the condition of at least one selected from the group consisting of the cornea, the crystalline lens, the eyeball, and the iris of the eye of the subject. For example, the irradiation light having a predetermined intensity tends to not reach the retina of the subject who has a high degree of opacity of the crystalline lens. Thus, the calibrator 19 may determine to increase the first reference intensity of the light to be emitted to the subject.

The calibrator 19 may calculate the illuminance of the irradiation light reaching the retina of the subject when each subject is irradiated with the reference irradiation light having a predetermined intensity in accordance with the intensity of the irradiation light, as well as the condition of at least one selected from the group consisting of the cornea, the crystalline lens, the eyeball, and the iris of the eye of the subject. The calibrator 19 may determine the intensity of the irradiation light to be emitted to the subject according to the relationship between the intensity of the irradiation light and the calculated illuminance. The calibrator 19 may store in advance data indicating the relationship between the intensity of the irradiation light, the conditions of the cornea, the crystalline lens, the eyeball, and the iris, and the illuminance of the irradiation light reaching the retina.

The state of at least one selected from the group consisting of the cornea, the crystalline lens, the eyeball, and the iris of the subject may be acquired from, for example, information of ophthalmic medical records of the subject. This allows emission of the irradiation light having an appropriate intensity for each subject.

### Time Manager 20

The time manager 20 measures the intensity of the irradiation light emitted to the eye of the subject by the irradiator 11 and the cumulative time spent irradiating the eye of the subject for each subject. For example, the time manager 20 may acquire information indicating the time from a timer (not illustrated) included in the light irradiation device 1.

The intensity of the irradiation light emitted to the eye of the subject is, for example, the intensity of the irradiation light when the eyelid of the subject is in the open state (first reference intensity) and the intensity of the irradiation light when the eyelid of the subject is in the closed state (second reference intensity). The cumulative time during which the irradiation light is emitted may be the cumulative time during which the irradiation light is emitted to the subject within a predetermined period such as one day, one month, or half a year, or may be the cumulative time during which the irradiation light is emitted in a single treatment.

### Outputter 21

The outputter 21 outputs the information indicating the intensity of the irradiation light and the information indicating the cumulative time during which the eyes of the subject is irradiated, which are measured by the time manager 20, to, for example, an external terminal, a server, or a storage device.

### Overview of Light Irradiation System 100

As described above, the light irradiation system 100 according to the present disclosure includes the irradiator 11 that irradiates the eye of the subject with the irradiation light having the predetermined wavelength band, and the shielding mechanism 12 that decreases the illuminance of the light included in light different from the irradiation light and having the wavelength band at least different from the predetermined wavelength band.

This allows the light irradiation system 100 to irradiate the eye of the subject with the irradiation light having the predetermined wavelength band and to avoid entrance of light having the wavelength band different from the predetermined wavelength band into the eyes of the subject. Using the light irradiation system 100, the predetermined wavelength band can be directed to the eye of the subject to effectively affect the mind and body of the subject.

The light irradiation system 100 may further include the imager 13 that captures an image of the subject and the display 14 that allows the medical worker or the subject to visually recognize the image of the eye of the subject captured by the imager 13. The light irradiation system 100 may further include the moving mechanism 15 that receives the operation for changing the position of the irradiator 11 relative to the position of the eye of the subject.

To obtain the effect on the mind and body of the subject effectively by irradiating light having the predetermined wavelength band, the irradiation light may be directed to the central fovea of the eye of the subject where cone cells are concentrated. Since the light irradiation system 100 includes the imager 13, the display 14, and the moving mechanism 15, the medical worker or the subject can move the position of the irradiator to an appropriate position using the moving mechanism 15 while checking the displayed image of the eye of the subject. Accordingly, the medical worker or the subject can efficiently direct the irradiation light to the central fovea.

The light irradiation system 100 may further include the light receiver 16 that detects the intensity of the return light of the irradiation light, and the eyelid opening/closing determiner 17 that determines the open/closed state of the eyelid of the subject based on the intensity of the return light. The light irradiation system 100 may further include the light intensity adjuster 18 that increases the intensity of the irradiation light when the eyelid of the subject is in the closed state, based on the determination result by the eyelid opening/closing determiner 17, compared to when the eyelid of the subject is in the open state.

In an example, the magnitude of the effect produced by irradiating the eye of the subject with the irradiation light having the predetermined wavelength band depends on the illuminance when the irradiation light reaches the retina of the eye of the subject. To obtain the effect by the irradiation light, an extreme decrease in the illuminance of the irradiation light reaching the retina of the subject may be avoided. However, since the irradiation light is blocked by the eyelid when the subject closes the eyelid, the illuminance may largely decrease when the irradiation light reaches the retina. With the light receiver 16, the eyelid opening/closing determiner 17, and the light intensity adjuster 18, the light irradiation system 100 can determine the open/closed state of the eyelid of the subject based on the intensity of the return light of the irradiation light, and adjust the intensity of the irradiation light based on the determination result of the open/closed state of the eyelid.

The light irradiation system 100 may further include the time manager 20 that measures the intensity of the irradiation light emitted to the eye of the subject and the cumulative time of irradiation for each subject, and the outputter 21 that outputs information indicating the measured intensity of the irradiation light and the cumulative time.

This allows the medical worker to manage the integrated illuminance of the irradiation light received by each subject and to evaluate the magnitude of the effect of irradiating the eye of the subject with the irradiation light for each subject.

The structure illustrated in FIG. 1 is merely an example and is not limited thereto. For example, the light irradiation system 100 may further include a server that receives the ID of each subject and manages the information measured by the time manager 20.

### Processing Executed by Light Irradiation System 100

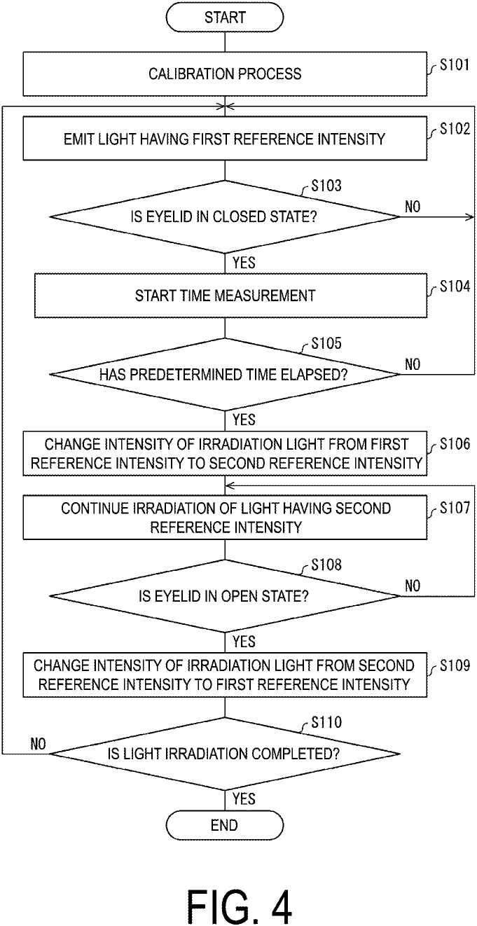

FIG. 4 is a flowchart illustrating a process flow executed by the light irradiation system 100 according to the present embodiment.

In S101, the calibrator 19 performs the above-described calibration processing to determine the first reference intensity and the second reference intensity which are the intensities of the irradiation light emitted by the irradiator 11 (calibration step).

In S102, the irradiator 11 irradiates light having the first reference intensity, and the process proceeds to S103 (light irradiation step).

In S103, the eyelid opening/closing determiner 17 determines whether the eyelid of the subject is in the closed state. If the eyelid opening/closing determiner 17 determines that the eyelid of the subject is in the closed state (YES in S103), the process proceeds to S104. If the eyelid opening/closing determiner 17 determines that the eyelid of the subject is not in the closed state, that is, is in the open state (NO in S103), the process returns to S102, and the irradiator 11 continues the irradiation of light having the first reference intensity.

In S104, when the eyelid is determined to be in the closed state in S103, the eyelid opening/closing determiner 17 then starts time measurement, and the process proceeds to S105. At this time, the time measurement may be performed by the time manager 20, for example.

In S105, the eyelid opening/closing determiner 17 determines whether the predetermined time has elapsed since the time measurement is started in S104 (time determination step). At this time, the time manager 20 may determine whether the predetermined time has elapsed. When the predetermined time has elapsed (YES in S105), the process proceeds to S106. If the predetermined time has not elapsed (NO in the S105), the process returns to S102, and the irradiator 11 continues emitting the light having the first reference intensity.

In S106, the light intensity adjuster 18 changes the intensity of the irradiation light emitted by the irradiator 11 from the first reference intensity to the second reference intensity, and the process proceeds to S107 (primary intensity changing step). That is, the eyelid of the subject having been closed for the predetermined time or longer indicates, for example, that the subject has fallen asleep. The irradiator 11 irradiates the light of the second reference intensity, which is higher than the first reference intensity. As a result, the light reaches the retina of the subject with the same illumination intensity as in the open state.

In S107, the irradiator 11 continues light irradiation at the second reference intensity, and the process proceeds to S108.

In S108, the eyelid opening/closing determiner 17 determines whether the eyelid of the subject is in the open state. If the eyelid opening/closing determiner 17 determines that the eyelid of the subject is in the open state (YES in S108), the process proceeds to S109. If the eyelid opening/closing determiner 17 determines that the eyelid of the subject is not in the open state, that is, in the closed state (NO in S108), the process returns to S107, and the irradiator 11 continues emitting the light having the second reference intensity.

In S109, the light intensity adjuster 18 changes the intensity of the irradiation light emitted by the irradiator 11 from the second reference intensity to the first reference intensity (second intensity changing step). In S110, the light irradiation system 100 determines whether to terminate the light irradiation, and if the end of the light irradiation is received (YES in S110), the irradiator 11 terminates the light irradiation. If the light irradiation system 100 receives non-termination of the light irradiation (NO in S110), the process returns to S102 and the irradiator 11 continues the processing from S102 to S110.

### Second Embodiment

Another embodiment of the present disclosure will be described below. For convenience of description, a member having the same function as that of a member described in the embodiments described above is denoted by the same reference sign, and description thereof will not be repeated.

In the first embodiment, the light irradiation device 1 provided as the wearable device has been described. In the present embodiment, the light irradiation device 1 of the installation-type is described with reference to FIGS. 5 and 6. FIG. 5 is a perspective view illustrating an example structure of a light irradiation system 100a according to a second embodiment. FIG. 6 illustrates a bottom surface of an example structure of the light irradiation system 100a according to the second embodiment. In the present embodiment, the light irradiation device 1a is the installation-type device.

As the same as or similar to the light irradiation device 1, the light irradiation device 1a includes an irradiator 11a and a shielding mechanism 12a. The light irradiation device 1a further includes the support 50 that supports the subject in a sitting or lying position. The irradiator 11a will be described later with reference to FIG. 6. The light irradiation device 1a may further include a display 14a and a controller 10a as illustrated in FIG. 5, although they are not essential. The light irradiation device 1a may further include an imager 13a, a moving mechanism 15a, and a light receiver 16a. The imager 13a, the moving mechanism 15a, and the light receiver 16a will be described later with reference to FIG. 6.

As illustrated in FIG. 5, the support 50 of the light irradiation device 1a is, for example, a bed. The shielding mechanism 12a is a half-pipe type mechanism in which an insertion hole 40 is formed as an inner hole therein. The insertion hole 40 is a portion surrounded by an inner peripheral surface 33 and the support 50, and the head of the subject is inserted through the hole.

A shielding portion 121 is provided at the entrance of the insertion hole 40 into which the head of the subject is inserted. The shielding portion 121 is provided on the same surface as an outer surface 32 of the shielding mechanism 12a and is joined to the end of the outer surface 32. The shielding portion 121 may be made of light shielding fabric that the subject can easily move the head in and out. Providing the shielding portion 121 can decrease the illuminance of the light reaching the eye of the subject and having the wavelength band different from the wavelength band of the irradiation light when irradiating the eye of the subject with the irradiation light having the predetermined wavelength band.

A display 14a corresponds to the display 14 in the first embodiment. In the present embodiment, the display 14a may be disposed on an outer peripheral surface 31 of the light irradiation device 1a.

The controller 10a corresponds to the controller 10 in the first embodiment and performs control so that the functions included in the light irradiation device 1a can be executed.

FIG. 6 is a schematic diagram illustrating an inner peripheral surface 33 of the light irradiation device 1a in FIG. 5 beyond the line of sight A as seen from the subject when the subject inserts the head into the insertion hole 40. The irradiator 11a, the imager 13a, the moving mechanism 15a, and the light receiver 16a are installed on the inner peripheral surface 33 of the shielding mechanism 12a.

The irradiator 11a corresponds to the irradiator 11 of the first embodiment. The irradiator 11a is disposed at a position opposite to the eye of the subject in the shielding mechanism 12a. Specifically, the irradiator 11a needs to be disposed on the inner peripheral surface 33 of the shielding mechanism 12a at a position opposite to the eye of the subject when the subject inserts the head into the insertion hole 40. The imager 13a corresponds to the imager 13 of the first embodiment. The image data captured by the imager 13a may be transmitted to the display 14a disposed on the outer peripheral surface 31. The imager 13a may be disposed in the vicinity of the irradiator 11a as illustrated in FIG. 6, and in this case, the position of the imager 13a may be changed together with the irradiator 11a using the moving mechanism 15a.

As illustrated in FIG. 6, the moving mechanism 15a may be a guide rail 152a that moves the irradiator 11a in the X-axis direction (left-right direction) and the Y-axis direction (vertical direction). Like the first embodiment, a direction parallel to the front-rear axis of the eyeball (that is, the axis connecting the cornea and the retina) is defined as the Z-axis direction, a direction parallel to the left-right axis (that is, the axis connecting the left ear and the right ear) is defined as the X-axis direction, and a direction parallel to the vertical axis (that is, the axis connecting the head and the side) is defined as the Y-axis direction. Although not illustrated in FIG. 6, the irradiator 11a may be held at a first end (corresponding to the first end 1511 in the first embodiment and FIG. 2) of a holding portion (not illustrated), and a second end (corresponding to the second end 1512 in the first embodiment and FIG. 2) of the holding portion may move on the guide rail 152a.

The light receiver 16a corresponds to the light receiver 16 of the first embodiment. The light receiver 16a may be provided in the vicinity of the irradiator 11a as illustrated in FIG. 6, or may be changed together with the irradiator 11a using the moving mechanism 15a.

### Example of Software Implementation

The control blocks (controller 10, 10a) of the light irradiation systems 100, 100a may be implemented by a logic circuit (hardware) formed in an integrated circuit (IC chip) or the like, or may be implemented by software.

In the latter case, the light irradiation systems 100, 100a include a computer that executes the instructions of a program, which is software that implements the functions. The computer includes, for example, one or more processors and a computer-readable recording medium that stores the above program. Then, in the computer, the processor reads the above program from the recording medium and executes the read program to achieve the object of the present disclosure. As the processor, a central processing unit (CPU) can be used, for example. As the recording medium, a “non-transitory tangible medium” such as, for example, a read only memory (ROM), a tape, a disk, a card, a semiconductor memory, a programmable logic circuit, and the like can be used. Additionally, a random access memory (RAM) for loading the above program may be further provided. The above program may be supplied to the computer via any transmission medium (communication network, broadcast wave, and the like) that can transmit the program. One aspect of the present disclosure may be implemented in the form of data signals embedded in a carrier wave in which the above program is embodied by electronic transmission.

In the present disclosure, the invention has been described above based on the various drawings and examples. However, the invention according to the present disclosure is not limited to each embodiment described above. That is, the embodiments of the invention according to the present disclosure can be modified in various ways within the scope illustrated in the present disclosure, and embodiments obtained by appropriately combining the technical means disclosed in different embodiments are also included in the technical scope of the invention according to the present disclosure. In other words, note that a person skilled in the art can easily make various variations or modifications based on the present disclosure. Note that these variations or modifications are included within the scope of the present disclosure.

### REFERENCE SIGNS

11, 11a Irradiator
12, 12a Shielding mechanism
13, 13a Imager
14, 14a Display
15, 15a Moving mechanism
16, 16a Light receiver
17 Eyelid opening/closing determiner
18 Light intensity adjuster
19 Calibrator
20 Time manager
21 Outputter

1. A light irradiation system comprising:
an irradiator configured to irradiate an eye of a subject with irradiation light having a predetermined wavelength band; and
a shielding mechanism configured to decrease illuminance of light that is included in light different from the irradiation light and that has a wavelength band different at least from the predetermined wavelength band.

2. The light irradiation system according to claim 1, further comprising:
an imager configured to capture an image of the eye of the subject;
a display configured to display the image of the eye of the subject captured by the imager; and
a moving mechanism configured to receive an operation for changing a position of the irradiator with respect to a position of the eye of the subject.

3. The light irradiation system according to claim 2, wherein
the moving mechanism is configured to change the position of the irradiator to a position at which the irradiation light is allowed to be directed toward a pupil of the eye of the subject.

4. The light irradiation system according to claim 1, further comprising:
a light receiver configured to detect an intensity of return light of the irradiation light;
an eyelid opening/closing determiner configured to determine an open/closed state of an eyelid of the subject based on the intensity of the return light; and
a light intensity adjuster configured to increase an intensity of the irradiation light when the eyelid of the subject is in a closed state to be higher than the intensity of the irradiation light when the eyelid of the subject is in an open state based on a determination result by the eyelid opening/closing determiner.

5. The light irradiation system according to claim 4, wherein
when the closed state of the eyelid of the subject continues for less than a predetermined time, the light intensity adjuster does not increase the intensity of the irradiation light.

6. The light irradiation system according to claim 5, wherein
the light intensity adjuster decreases the intensity of the irradiation light when the eyelid of the subject is in the open state subsequent to the closed state continued for the predetermined time or longer.

7. The light irradiation system according to claim 4, further comprising:
a calibrator configured to
continuously irradiate the eye of the subject with the irradiation light having a predetermined intensity for a certain period of time,
acquire a first intensity of the return light detected when the eyelid of the subject is in the open state and a second intensity of the return light detected when the eyelid of the subject is in the closed state, and
determine, based on a comparison result between the first intensity and the second intensity, a first reference intensity of the irradiation light when the eyelid of the subject is in the open state and a second reference intensity of the irradiation light when the eyelid of the subject is in the closed state.

8. The light irradiation system according to claim 7, wherein
the calibrator determines the first reference intensity based on a condition of at least one selected from the group consisting of a cornea, a crystalline lens, an eyeball, or an iris of the eye of the subject.

9. The light irradiation system according to claim 7, wherein
the light intensity adjuster adjusts the intensity of the irradiation light by applying the first reference intensity and the second reference intensity determined by the calibrator.

10. The light irradiation system according to claim 1, wherein
an intensity of the irradiation light is 4 Lux to 100 Lux.

11. The light irradiation system according to claim 1, further comprising:
a time manager configured to measure an intensity of the irradiation light emitted to the eye of the subject and cumulative time of irradiation; and
an outputter configured to output information indicating a measured intensity of the irradiation light and information indicating the a measured cumulative time of irradiation.

12. The light irradiation system according to claim 1, wherein
the predetermined wavelength band is either from 450 nm to 600 nm and/or from 350 nm to 410 nm.

13. A wearable device worn on a head of a subject, comprising:
the light irradiation system of claim 1.

14. An installation-type light irradiation device comprising:
a support configured to support a subject in a sitting or lying position;
an irradiator configured to irradiate an eye of the subject with irradiation light having a predetermined wavelength band; and
a shielding mechanism configured to decrease illuminance of light reaching the eye of the subject, included in light different from the irradiation light, and having a wavelength band different at least from the predetermined wavelength band, wherein
the irradiator is disposed in the shielding mechanism at a position opposite to the eye of the subject.

15. A light irradiation method comprising:
calibrating by
irradiating an eye of a subject with irradiation light having a predetermined intensity for a certain period of time,
acquiring a first intensity of return light detected when an eyelid of the subject is in an open state and a second intensity of the return light detected when the eyelid of the subject is in a closed state, and
determining a first reference intensity of the irradiation light when the eyelid of the subject is in the open state and a second reference intensity of the irradiation light when the eyelid of the subject is in the closed state based on a comparison result between the first intensity and the second intensity;
emitting the irradiation light having the first reference intensity;
changing an intensity of the irradiation light from the first reference intensity to the second reference intensity upon a determination as to whether the eyelid of the subject is in the closed state and whether a predetermined time has passed in the closed state; and
changing the intensity of the irradiation light from the second reference intensity to the first reference intensity upon determination as to whether the eyelid of the subject is in the open state.
